SEQUENCE LISTING (1 sequence)

SEQ ID NO: 1 - protein, 269 aa, Rhizomucor miehei
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSVLDHLSYFGINTGLCT

(19) United States
(12) Patent Application Publication
(10) Pub. No.: US 2024/0076648 A1
(43) Pub. Date: Mar. 7, 2024

(54) POLYSTYRENE/DIVINYLBENZENE PARTICLES FOR LIPASE IMMOBILIZATION

(71) Applicant: Novozymes A/S, Bagsvaerd (DK)

(72) Inventor: Pavle Andric, Copenhagen (DK)

(73) Assignee: Novozymes A/S, Bagsvaerd (DK)

(21) Appl. No.: 18/547,619

(22) PCT Filed: Feb. 28, 2022
(86) PCT No.: PCT/EP2022/055000

§ 371 Date: Aug. 23, 2023

(30) Foreign Application Priority Data
Feb. 26, 2021 (EP) 21159635.8

(51) Int. Cl. C12N 11/082; C12N 9/20; C12P 7/6458

(52) U.S. Cl. CPC C12N 11/082; C12N 9/20; C12P 7/6458; C12Y 301/01003

(57) ABSTRACT

The invention provides enzyme particles comprising an immobilized 1,3 specific lipase and a copolymer of styrene and divinylbenzene. The particles are suitable for enzymatic interesterification of triglycerides, and subsequent separation of the enzyme and triglycerides by filtration.

### REFERENCE TO A SEQUENCE LISTING

This application contains a Sequence Listing in computer readable form, which is incorporated herein by reference.

### FIELD OF THE INVENTION

The present invention relates to polystyrene/divinylbenzene particles for immobilization of positionally 1,3 site specific lipases. The resulting lipase particles are useful for preparing symmetric triglycerides in enzymatic esterification/interesterification.

### BACKGROUND

Immobilization of lipolytic enzymes has been known for many years. An immobilized enzyme product may be used in enzymatic modification of an organic compound such as in organic synthesis processes, vegetable oil interesterification, biodiesel production etc.

Enzyme immobilization is the attachment of an enzyme protein on a carrier on which the enzyme is fixed, yet functional, where the enzyme is not released into the liquid (washed out) to which it is contacted. The most commonly immobilized enzymes are glucose isomerase used for isomerization reactions, and lipase used for, e.g., interesterification of vegetable oils and organic synthesis.

For use in non-aqueous solutions, lipases can be immobilized on a number of different porous, inorganic carriers by absorption of an aqueous solution of lipase into the pore volume of the carrier, or by adsorption to the surface of the carrier, or by a combination of both adsorption and absorption followed by water removal by drying.
JP 5-292965A discloses an immobilized lipase and a method for preparing it.
WO 95/22606 describes an immobilization process based on a granulation process.
WO 99/33964 describes an immobilization process wherein the enzyme is applied to a particulate porous carrier.

Immobilized enzymes are known to be used in both continuous and batch enzymatic reactions within a variety of industrial applications, including wastewater treatment, production of pharmaceuticals, high fructose corn syrup production, vegetable oil processing and synthesis of chemicals.

### SUMMARY OF THE INVENTION

In a first aspect, the present invention provides (a plurality of) enzyme particles, comprising
(a) 50-99% w/w of a copolymer of styrene and divinylbenzene; and
(b) 1-20% w/w active enzyme protein of a 1,3 site specific lipase;
wherein the 1,3 specific lipase has at least 80% amino acid sequence identity to SEQ ID NO:1.

In another aspect of the invention is provided methods for enzymatic esterification and interesterification, comprising contacting mixtures of free fatty acid/alcohol or triglycerides with the enzyme particles of the invention.

Other aspects and embodiments of the invention are apparent from the description and examples.

Unless otherwise indicated, or if it is apparent from the context that something else is meant, all percentages are percentage by weight (% w/w).

### DETAILED DESCRIPTION

We have found that copolymers of styrene and divinylbenzene provides an advantageous polymeric support for immobilization of 1,3-specific lipases (such as Rhizomucor miehei lipase). The performance of the immobilized 1,3-specific lipases is surprisingly good for production of symmetric triglycerides.

Polymethacrylate-based particles are widely used for immobilization of lipases by adsorption, because of the high-affinity of these materials for lipases. Likewise, polystyrene is not considered as a particularly suitable material for this type of immobilization of lipases, because of a lower affinity, and thus a lower adsorption, of lipases. This is unfortunate because polystyrene is a relatively cheap material, as opposite to the relatively expensive methacrylic adsorbents.

We hereby propose to use a low-cost polystyrene-based polymer support for immobilization of 1,3-specific lipases. This material has larger pores that allow for enzyme penetration into the particle, and at the same type provide good mass-transfer conditions for the subsequent enzymatic reaction, as well as favorable water-free conditions due to the high-hydrophobicity of the material. The benefits are particularly seen during production of symmetric triglycerides, like stearic-oleic-stearic and oleic-palmitic-oleic triglycerides.

The use of lipase catalyzed interesterification of fats/oils is well established. The reaction takes place in columns with a height of 1-5 meters filled with immobilized enzyme with a typical particle size of 300-1200 μm, where the oil is pumped through a set of columns with the total holding time required to achieve a certain conversion.

A highly desirable property of using the polystyrene-based polymer support of the invention is an improved mechanical strength of the immobilized lipase particles. When lipase particles are packed in high columns, the particles at the bottom of the column will be exposed to the accumulated weight of the particles above. This may lead to a collapse of the particles at the bottom and a consequential reduced oil flow through the column. Particles made of polystyrene-based polymer supports are particularly strong compared to methacrylate-based polymer supports. Due to its hydrophobic nature, polystyrene-based polymer supports are also highly resistant to swelling compared hydrophilic materials like methacrylate-based polymer supports. Swelling of immobilized lipase particles also causes reduced oil flow through the column.

Unless otherwise indicated, all percentages are indicated as percent by weight (% w/w) throughout the application.

### Enzyme Particles

The particles of the invention consist of an immobilized positionally 1,3 site specific lipase and a polymeric material comprising, or consisting of, copolymers of styrene and divinylbenzene. The particles may be encapsulated in oil or fat; e.g. to form an oily powder or a slurry/suspension.

The particles are preferably porous. The pore volume may correspond to an oil uptake of at least 0.5 gram of oil per gram of particles, particularly at least 1 gram of oil per gram of particles. It may have a surface area of 5-1000 m²/g, 10-1000 m²/g, in particular 10-700 m²/g, more particularly 10-500 m²/g.

The particles may have a volume-based particle size (D 50) of 200-1200 μm, preferably 200-1000 μm, and more preferably 300-900 μm. The particle size is measured with a laser diffraction particle size analyzer.

The particles may comprise the polymeric material in an amount of more than 60% w/w, preferably more than 80% w/w.

In order to make a final product with low dust properties and/or improved compatibility with the process in which it will be used (e.g., an interesterification process), the resulting particles can be sprayed with oil, or be blended with oil to obtain an oily powder or a slurry/suspension that encapsulates the particles in oil. The oil may be a plant derived oil, such as sunflower oil or another oil which is compatible with the process in which the particles will be used. If only a small amount of oil is used, the particles can be agglomerated into larger particles that can substantially reduce the amount of dust. The oil-encapsulated particles can also be dried subsequently.

The particles can also be sprayed or blended with fat to produce a solid block containing fat and small particles or processed through extrusion and pelletizing equipment to obtain large pellets, which may also include added fat as a ‘vehicle’. Such particles can subsequently be coated with a preservation agent, for example a powderized preservation agent.

Dust is defined as particles with an aerodynamic diameter less than 50 μm. In aerosol science, it is generally accepted that particles with an aerodynamic diameter higher than 50 μm do not commonly remain airborne for very long. In this context, the aerodynamic diameter is defined as “the diameter of a hypothetical sphere of density 1 g/cm 3 having the same terminal settling velocity in calm air as the particle in question, regardless of its geometric size, shape and true density.” (WHO, 1997).

Lipase

The lipase to be immobilized according to the invention is a positionally 1,3 site specific lipase (sn-1,3-specific lipase) classified under the Enzyme Classification number EC 3.1.1.3 (Triacylglycerol lipase).

Examples of positionally 1,3 site specific lipases for use in the invention include Rhizomucor miehei lipase (see also WO 2015/181119), Rhizopus oryzae lipase, Rhizopus delemarilipase, Malbranchea cinnamomea lipase (see also Biotechnol Appl Biochem, 63(4), pp 471-478 (2016)), Thermomyces lanuginosus lipase, and variants thereof with more than 80% amino acid sequence identity (exhibiting 1,3 site specific lipase activity).

In an embodiment, the lipase is a Rhizomucor miehei lipase, or a variant thereof having more than 80% (or more than 90% or 95%) amino acid sequence identity to SEQ ID NO:1. In a preferred embodiment, the lipase is a Rhizomucor miehei lipase, such as SEQ ID NO:1.

In an embodiment of the invention, the enzyme particle comprises the 1,3 site specific lipase in an amount of 1-20% w/w active enzyme protein. In another embodiment, the enzyme particle comprises the 1,3 site specific lipase in an amount of 1-20% w/w active enzyme protein. In a particular embodiment, the enzyme particle comprises the 1,3 site specific lipase in an amount of 1-10% w/w active enzyme protein.

Active enzyme protein is defined herein as the amount of lipase protein(s), which exhibits lipase activity. This can be determined using an activity based analytical enzyme assay. In such assays, the enzyme typically catalyzes a reaction generating a colored compound. The amount of the colored compound can be measured and correlated to the concentration of the active enzyme protein. This technique is well-known in the art.

1,3 site specific lipases catalyze the spontaneous acyl migration from position 2 to positions 1 or 3.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the nobrief option must be specified in the command line.

The output of Needle labeled “longest identity” is calculated as follows:

(Identical Residues×100)/(Length of Alignment−Total Number of Gaps in Alignment).

### Polymeric Material

The particles of the invention comprise copolymers of styrene and divinylbenzene, which provide a hydrophobic surface for adsorption of the 1,3 site specific lipase.

The copolymers of styrene and divinylbenzene may also include residues of other monomers, like methyl methacrylate, ethyl methacrylate, butyl methacrylate, octadecyl methacrylate, methyl acrylate, ethyl acrylate, butyl acrylate, and octadecyl acrylate.

In an embodiment, the copolymers comprise more than 90% w/w of styrene and divinylbenzene residues (units). Preferably, the copolymers consist of styrene and divinylbenzene residues.

In an embodiment, the copolymer of styrene and divinylbenzene comprises styrene and divinylbenzene residues in a ratio of from 100:1 to 2:1; preferably in a ratio of from 50:1 to 3:1; and more preferably in a ratio of from 20:1 to 3:1.

The copolymers may be provided in the form of a macro-porous material, such as a macro-porous polymer matrix, or macro-porous polymer beads.

Such macro-porous materials may have a copolymer content of at least 50% w/w, preferably at least 80% w/w, and more preferably at least 90% w/w. Most preferably, the material consists of copolymers of styrene and divinylbenzene. The material may have an average particle size in the range of 200-1000 μm, such as 300-900 μm.

In a preferred embodiment, the hydrophobic polymers are provided as macro-porous particles consisting of copolymers of styrene and divinylbenzene with an average particle size in the range of 200-1000 μm.

The enzyme particles of the invention may comprise the copolymers of styrene and divinylbenzene in an amount of 50-99% w/w, preferably 80-99% w/w.

### Uses of the Particles

Particles comprising immobilized 1,3 site specific lipases, according to the invention, have potential applications in a wide range of enzymatic employed processes such as in the production of pharmaceuticals, specialty commodity chemicals, and vegetable oil processing.

Immobilized enzymes prepared in the context of the invention may be used for hydrolysis, synthesis or modification of organic substances. The hydrolysis, synthesis or modification preferably takes place in a medium essentially devoid of free water.

Accordingly, the invention encompasses a process for enzymatic modification of an organic compound comprising contacting in a reaction medium said organic compound with an immobilized enzyme product according to the invention.

The immobilized enzyme of the present invention may be used for enzymatic modification of an organic compound comprising contacting in a reaction medium said organic compound with an immobilized enzyme produced by the process of the invention.

In a particular embodiment of the present invention the modification is an esterification reaction comprising contacting a first reactant which is a carboxylic acid and a second reactant which is an alcohol with an immobilized lipase of the invention. The carboxylic acid may be selected from but not limited to the group consisting of fatty acids, lactic acid, benzoic acid, acrylic acid and methacrylic acid and the alcohol may be selected from but not limited to the group consisting of methanol, ethanol, isopropanol, polyols such as glycerol, sorbitol, isosorbide, xylitol, glucosides such as ethyl and methyl glucosides, neopentyl alcohol and propylene glycol.

The modification may be a chiral resolvation including an enantioselective synthesis or hydrolysis of carboxylic acid ester or amides; an aldol condensation reaction between two aldehydes; or an epoxidation of olefinic groups by percarboxylic acid produced in situ by the immobilized enzyme.

The modification may be a polyesterification reaction wherein the organic compound to be modified is a hydroxycarboxylic acid or oligomers of such compound e.g. lactic acid or 3-hydroxypropanoic acid. Or the carboxylic acid is a dicarboxylic acid selected from the group consisting of adipic acid, succinic acid, fumaric acid, 2,5-furandicarboxylic acid, glucaric acid, terephthalic acid and isophthalic acid, and the second reactant is selected from the group consisting of polyols such as 1,4-butanediol, 1,6-hexanediol, glycerol, sorbitol, isosorbide, neopentyl alcohol, or propylene glycol.

In another particular embodiment the modification is a ring opening polymerization reaction comprising contacting a lactone with an immobilized lipase produced by the present process. Prepared polymers may be homo or hetero polymers.

The modification may be a transesterification reaction comprising contacting a first reactant which is a carboxylic acid ester and a second reactant which is an alcohol with an immobilized lipase produced by the present process.

The modification may be an interesterification reaction comprising contacting a first reactant which is a carboxylic acid ester and a second reactant which is a second carboxylic acid ester with an immobilized lipase produced by the present process. In a more particular embodiment the modification is an interesterification reaction comprising contacting a first reactant which is a polycarboxylic acid ester and a second reactant which is a second poly-carboxylic acid ester, with an immobilized lipase of the invention.

By interesterification of two different fats/oils the change in fatty acid positions resulting from the interesterification will impact the melting profile of the oils/fat mixture. This is measured by NMR and expressed as the percentage of solid fat at a given temperature in the typical range 10° C.-40° C. Examples of components are coconut fat and palm stearine.

The carboxylic acid ester may be selected from but not limited to the group consisting of alkyl esters of fatty acids, lactic acid, glucaric acid, benzoic acid, acrylic acid, methacrylic acid and wherein the alkyl may be methyl, ethyl, butyl and the alcohol may be selected from the group consisting of but not limited to methanol, ethanol, isopropanol, polyols such as glycerol, alkyl glucosides, such as ethyl glucoside or methyl glucoside, sorbitol, silicone and silicone derivatives, isosorbide, neopentyl alcohol and propylene glycol.

The modification may be a hydrolysis or synthesis producing an enantiopure compound; an amidation reaction comprising contacting a first reactant which is a carboxylic acid and a second reactant which is an amine with an immobilized lipase of the invention.

In a particular embodiment, the modification is an epoxidation reaction comprising in situ production of epoxidation agent with an immobilized enzyme produced by the present process.

In an embodiment of the present invention an immobilized lipase enzyme is used for an esterification, transesterification or interesterification process in a medium essentially devoid of free water. The transesterification may be used for fatty acid substitution, comprising contacting a first reactant and a second reactant with said immobilized lipase by which a substitution reaction occurs.

The first reactant may be a fatty acid ester, preferably a triglyceride or a mixture of triglycerides.

The second reactant may be another fatty acid ester different from the first reactant, preferably a triglyceride or a mixture of triglycerides. Further the second reactant may be an alcohol or a free fatty acid.

The medium in this preferred embodiment of the invention comprises an organic solvent, or it may consist essentially of triglycerides.

Said use of the invention may be applied in production of food products e.g. margarine or cocoa-butter substitutes, for production of esters for e.g. cosmetics, biofuel, etc.

Processes

The invention also provides a process for conducting a reaction catalyzed by the 1,3 site specific lipase particles of the invention, comprising:
a) preparing a reaction mixture comprising reactants for the reaction, and
b) contacting the reaction mixture with the immobilized 1,3 site specific lipase particles at conditions which are effective for conducting the reaction.

The contact may be done by passing the reaction mixture through a packed-bed column of the immobilized 1,3 site specific lipase, a continuously stirred tank reactor holding the immobilized lipase, a moving bed reactor where the movement of the packed bed of immobilized enzyme is either co-current or counter-current to the reaction mixture, in a batch reactor, optionally with stirring or in any other type of reactor or combination of reactor in which the desired reaction can be carried out.

The reactants may comprise a fatty acyl donor and an alcohol, and the reaction may form a fatty acid alkyl ester.

The reactants may comprise at least two triglycerides, and the reaction may form different triglycerides. Thus, the reaction may be carried out for a time sufficient to change the melting properties of the mixture of triglycerides.

The present invention is further described by the following numbered embodiments:

Embodiment 1. An (a plurality of) enzyme particle, comprising
(a) 50-99% w/w of a copolymer of styrene and divinylbenzene; and
(b) 1-20% w/w active enzyme protein of a 1,3 specific lipase from enzyme class EC 3.1.1.3.

Embodiment 2. The enzyme particle of embodiment 1, which comprises the copolymer of styrene and divinylbenzene in an amount of 80-99% w/w.

Embodiment 3. The enzyme particle of any of embodiments 1-2, wherein the copolymer of styrene and divinylbenzene comprises styrene and divinylbenzene units in a ratio of from 100:1 to 2:1.

Embodiment 4. The enzyme particle of any of embodiments 1-3, wherein the copolymer of styrene and divinylbenzene comprises styrene and divinylbenzene units in a ratio of from 50:1 to 3:1.

Embodiment 5. The enzyme particle of any of embodiments 1-4, wherein the copolymer of styrene and divinylbenzene comprises styrene and divinylbenzene units in a ratio of from 20:1 to 3:1.

Embodiment 6. The enzyme particle of any of embodiments 1-5, which comprises the 1,3 specific lipase in an amount of 1-15% w/w active enzyme protein.

Embodiment 7. The enzyme particle of any of embodiments 1-6, which has a surface area of 5-1000 m²/g.

Embodiment 8. The enzyme particle of any of embodiments 1-7, which has a surface area of 10-750 m²/g.

Embodiment 9. The enzyme particle of any of embodiments 1-8, which has a pore volume corresponding to an oil uptake of at least 0.5 gram of oil per gram of particles, particularly at least 1 gram of oil per gram of particles.

Embodiment 10. The enzyme particle of any of embodiments 1-9, which has an average pore size of 0.05-0.1 μm.

Embodiment 11. The enzyme particle of any of embodiments 1-10, which has an average pore size of 0.1-0.5 μm.

Embodiment 12. The enzyme particle of any of embodiments 1-11, which have an average diameter of 200-1200 μm.

Embodiment 13. The enzyme particle of any of embodiments 1-12, which have an average diameter of 200-1000 μm.

Embodiment 14. The enzyme particle of any of embodiments 1-13, which have an average diameter of 300-900 μm.

Embodiment 15. The enzyme particle of any of embodiments 1-14, wherein the 1,3 specific lipase is a Rhizomucor lipase.

Embodiment 16. The enzyme particle of any of embodiments 1-15, wherein the 1,3 specific lipase is a Rhizomucor miehei lipase.

Embodiment 17. The enzyme particle of any of embodiments 1-16, wherein the 1,3 specific lipase has at least 60% amino acid sequence identity to SEQ ID NO:1.

Embodiment 18. The enzyme particle of any of embodiments 1-17, wherein the 1,3 specific lipase has at least 80% amino acid sequence identity to SEQ ID NO:1.

Embodiment 19. The enzyme particle of any of embodiments 1-18, wherein the 1,3 specific lipase has at least 90% amino acid sequence identity to SEQ ID NO:1.

Embodiment 20. The enzyme particle of any of embodiments 1-19, wherein the 1,3 specific lipase has at least 95% amino acid sequence identity to SEQ ID NO:1.

Embodiment 21. The enzyme particle of any of embodiments 1-20, wherein the 1,3 specific lipase has the amino acid sequence shown in SEQ ID NO:1.

Embodiment 22. The enzyme particle of any of embodiments 1-14, wherein the 1,3 specific lipase is a Rhizopus lipase.

Embodiment 23. The enzyme particle of any of embodiments 1-14, wherein the 1,3 specific lipase is a Rhizopus oryzae lipase.

Embodiment 24. The enzyme particle of any of embodiments 1-14, wherein the 1,3 specific lipase is a Thermomyces lipase.

Embodiment 25. The enzyme particle of any of embodiments 1-14, wherein the 1,3 specific lipase is a Thermomyces lanuginosus lipase.

Embodiment 26. The enzyme particle of any of embodiments 1-14, wherein the 1,3 specific lipase is a Malbranchea lipase.

Embodiment 27. The enzyme particle of any of embodiments 1-14, wherein the 1,3 specific lipase is a Malbranchea cinnamomea lipase.

Embodiment 28. The enzyme particle of any of embodiments 1-27, wherein the copolymer of styrene and divinylbenzene comprise more than 90% of styrene and divinylbenzene units.

Embodiment 29. The enzyme particle of any of embodiments 1-28, wherein the copolymer of styrene and divinylbenzene consists of styrene and divinylbenzene units.

Embodiment 30. A powder or a slurry/suspension comprising the enzyme particle of any of embodiments 1-29 and at least 10% oil or fat.

Embodiment 31. A method for enzymatic interesterification, comprising contacting a mixture of triglycerides and free fatty acid(s) or free fatty acid ester(s) with the enzyme particle according to any of embodiments 1-29.

Embodiment 32. A method for enzymatic esterification comprising contacting a carboxylic acid and an alcohol with the enzyme particle of any of embodiments 1-29; preferably the reaction is carried out in a column.

Embodiment 33. The method of embodiment 32, which is a method for production of symmetric triglycerides by enzymatic esterification.

Embodiment 34. Use of the enzyme particle of any of embodiments 1-29 in an esterification reaction, comprising contacting a carboxylic acid and an alcohol with the enzyme particle for production of symmetric triglycerides; preferably the reaction is carried out in a column.

### EXAMPLES

The present invention is further described by the following example which should not be construed as limiting the scope of the invention.

Chemicals were commercial products of at least reagent grade. The Rhizomucor miehei lipase has the amino acid sequence shown in SEQ ID NO:1.

### Example 1

Immobilization of Rhizomucor miehei lipase was carried out by adsorption on two types of particles/carriers using the same enzyme concentration in the adsorption step (grams of active enzyme protein added per gram of carrier material). The two types of particles/carriers were:

(a) a macroporous polystyrene resin/adsorbent (co-polymer with divinylbenzene), and

(b) a methacrylate resin/adsorbent (co-polymer with divinylbenzene).

This method is commonly used and well-known to a person skilled in the art. Briefly, a liquid lipase concentrate was mixed with the solid carrier material and allowed to remain in contact with the carrier material for 1-2 days.

After filtration, and washing with water, the produced immobilized enzyme particles (lipase on styrene/divinylbenzene and methacrylate/divinylbenzene, respectively) were separated from the supernatant, containing residual non-adsorbed enzyme, and dried.

The amount of residual enzyme in the supernatant was measured, and it indicated that immobilization on methacrylate/divinylbenzene particles was significantly more efficient, resulting in 5-10 times lower concentration of residual enzyme in the supernatant. Thus, the amount of enzyme adsorbed on the styrene/divinylbenzene particles was significantly lower than the amount adsorbed on methacrylate/divinylbenzene particles. The non-adsorbed enzyme from the supernatant was recovered.

The two types of immobilized enzyme particles were used to perform an interesterification reaction between triglyceride (triolein, 000) and stearic acid, in order to produce symmetric triglyceride (stearic-oleic-stearic, SOS).

The reactions were carried out by contacting the immobilized enzymes with reactants in a fixed-bed reactor. Reaction was performed at 75° C., by feeding reactants continuously to the reactor (55%/45% stearic acid:triglyceride mix), and the composition of the outlet from reactor (product stream) was measured for SOS and SSS concentration, by gas chromatography.

Briefly, the procedure for preparing the feed blend and pH adjustment was as follows:
1. Stearic acid was heated in a bottle in 85° C. oven/water bath (around 3 hours).
2. High oleic sunflower oil was added and bottle purged with nitrogen before cap tight.
3. Re-incubate the oil blend in 75° C. oven/water bath (around 1 hour).
4. Mix the oil blend with high-shear mixer at 24000 rpm for 30 seconds.
5. Add in caustic while high shear mixing (to prevent solidification).
6. Store in well capped bottle and blanket with nitrogen.

The performance of the polystyrene/divinylbenzene particles were found to be at least as good as the methacrylate/divinylbenzene particles, as measured by formation of SOS product, and SSS by-product. Interesterification using both types of particles yielded>25% of SOS, and about 1-1.1% of SSS.

Since the performance of the two types of particles was equal, the styrene/divinylbenzene immobilized lipase performance was about 5-10 times better than the commonly used methacrylate/divinylbenzene immobilized lipase.

### Example 2

A reaction was carried out with the two types of immobilized lipase particles used in Example 1. The reaction was run at 75° C. in column mode (continuous), under the following conditions:
2 g of immobilized enzyme (dry packing);
an oil blend consisting of stearic acid: high oleic sunflower oil in a weight ratio of 55:45; and
water and 200 ppm NaOH (4N)+0.1% added.

Conversion to SOS (stearic-oleic-stearic) and SSS (stearic-stearic-stearic) was measured.

At an oil blend flow rate of 3 g/g/h, the conversion to SOS (the desired product) kept increasing, while SSS (unwanted byproduct) remained constant for polystyrene/divinylbenzene particles; while for methacrylate/divinylbenzene particles, the conversion to SSS kept increasing at the same rate as SOS.

This result shows the desired selectivity of the polystyrene/divinylbenzene particle towards SOS, using the same type of immobilized lipase.

1-15. (canceled)

16. An enzyme particle, comprising:
(a) 50-99% w/w of a copolymer of styrene and divinylbenzene; and
(b) 1-20% w/w active enzyme protein of a 1,3 specific lipase from enzyme class EC 3.1.1.3;
wherein the 1,3 specific lipase has at least 80% amino acid sequence identity to SEQ ID NO: 1.

17. The enzyme particle of claim 16, wherein the copolymer of styrene and divinylbenzene is in an amount of 80-99% w/w.

18. The enzyme particle of claim 16, wherein the copolymer of styrene and divinylbenzene comprises styrene and divinylbenzene units in a ratio of from 100:1 to 2:1.

19. The enzyme particle of claim 16, wherein the copolymer of styrene and divinylbenzene comprises styrene and divinylbenzene units in a ratio of from 50:1 to 3:1.

20. The enzyme particle of claim 16, wherein the 1,3 specific lipase is in an amount of 1-15% w/w active enzyme protein.

21. The enzyme particle of claim 16, wherein the particle has a surface area of 5-1000 m²/g.

22. The enzyme particle of claim 16, wherein the particle has a surface area of 10-750 m²/g.

23. The enzyme particle of claim 16, wherein the particle has an average pore size of 0.05-0.1 μm.

24. The enzyme particle of claim 16, wherein the particle has an average pore size of 0.1-0.5 μm.

25. The enzyme particle of claim 16, wherein the particle has a pore volume corresponding to an oil uptake of at least 0.5 gram of oil per gram of particles.

26. The enzyme particle of claim 16, wherein the particle has a pore volume corresponding to an oil uptake of at least 1 gram of oil per gram of particles.

27. The enzyme particle of claim 16, wherein the particle has an average diameter of 200-1200 μm.

28. The enzyme particle of claim 16, wherein the particle has an average diameter of 300-900 μm.

29. The enzyme particle of claim 16, wherein the 1,3 specific lipase is a Rhizomucor lipase.

30. The enzyme particle of claim 16, wherein the 1,3 specific lipase is a Rhizomucor miehei lipase.

31. The enzyme particle of claim 16, wherein the 1,3 specific lipase has at least 90% amino acid sequence identity to SEQ ID NO: 1.

32. The enzyme particle of claim 16, wherein the 1,3 specific lipase has at least 95% amino acid sequence identity to SEQ ID NO: 1.

33. 11. The enzyme particle of claim 16, wherein the 1,3 specific lipase has the amino acid sequence of SEQ ID NO: 1.

34. The enzyme particle of claim 16, wherein the copolymer of styrene and divinylbenzene comprises more than 90% of styrene and divinylbenzene units.

35. The enzyme particle of claim 16, wherein the copolymer of styrene and divinylbenzene consists of styrene and divinylbenzene units.

36. A powder or a slurry/suspension comprising the enzyme particle of claim 16 and at least 10% oil or fat.

37. A method for enzymatic interesterification, comprising contacting a mixture of triglycerides and free fatty acid(s) or free fatty acid esters with the enzyme particle of claim 16.
